(19) United States
(12) Patent Application Publication
(10) Pub. No.: US 2014/0194748 A1
(43) Pub. Date: Jul. 10, 2014

(54) IMAGING DEVICE

(71) Applicant: OLYMPUS CORPORATION, Tokyo (JP)

(72) Inventors: Yusuke Yamamoto, Kawasaki-shi (JP); Kosei Tamiya, Tokyo (JP)

(73) Assignee: OLYMPUS CORPORATION, Tokyo (JP)

(21) Appl. No.: 14/137,242

(22) Filed: Dec. 20, 2013

(30) Foreign Application Priority Data
Jan. 8, 2013 (JP) 2013-000912

(51) Int. Cl. A61B 5/00

(52) U.S. Cl. CPC A61B 5/4887; A61B 5/0059
USPC 600/473

(57) ABSTRACT

A color filter has a transmission band in a visible region and an infrared region. A first substrate is arranged below the color filter and has a first photoelectric conversion element which outputs a first signal charge according to an amount of exposure of a light passing through the color filter. A second substrate has a second photoelectric conversion element outputting a second signal charge according to an amount of exposure of a light, having sensitivity in at least the infrared region, which passes through the first substrate, and is arranged on a surface on an opposite side to a light-receiving surface of the first substrate. A signal read-out circuit reads out the first signal charge as a first electrical signal, and reads out the second signal charge as a second electrical signal.

### BACKGROUND OF THE INVENTION

1. Field of the Invention

The present invention relates to an imaging device. Priority is claimed on Japanese Patent Application No. 2013-000912, filed Jan. 8, 2013, the contents of which are incorporated herein by reference.

2. Description of Related Art

In recent years, endoscope systems and vein authentication systems which detect cancer of biological tissue or a vein pattern of a finger by irradiating an object such as biological tissue or a finger with infrared light in addition to visible light, and by using a visible light image and an infrared light image which pass through the object or are reflected from the object, have been widely used.

For example, as disclosed in “the Entirety of ICG Fluorescence Navigation Surgery” written by Mitsuo Kusano, Inter Medica Co., Ltd., Nov. 20, 2008, endoscope systems perform not only an ordinary observation using visible light but also a specific observation using infrared light. The endoscope system administers in advance a fluorescent material such as indocyanine green (ICG), which has a predilection for a focus such as cancer, is excited in an infrared region, and emits fluorescence, into the body of an object to be inspected, and irradiates the object with excitation light that excites the fluorescent material to thereby detect fluorescence from the fluorescent materials that accumulate in a focus portion. Since strong fluorescence is radiated from the focus portion, the presence or absence of a lesion is determined from the brightness of a fluorescence image.

In addition, as disclosed in Japanese Unexamined Patent Application, First Publication No. 2010-92494, vein authentication systems detect a vein pattern by irradiating a finger with visible light and infrared light and by using the infrared light that passes through the finger and the visible light that is reflected at the finger. In a technique disclosed in Japanese Unexamined Patent Application, First Publication No. 2010-92494, the vein pattern is detected with a high level of accuracy by performing arithmetic processing using an infrared light image that is obtained by imaging dirt, wrinkles, or the like of a finger together with the vein pattern, and a visible light image that is reflected by a surface of the finger and is obtained by imaging dirt, wrinkles, or the like of the surface of the finger.

Next, there is described a configuration of an apparatus used to detect a visible light image and an infrared light image in a vein authentication system. FIG. 21 is a schematic diagram illustrating a configuration of a vein authentication system, known in the related art, which detects a vein pattern by detecting a visible light image and an infrared light image. A vein authentication system 1000 includes an infrared light source 1001, visible light sources 1002 and 1003, a dichroic mirror 1004, a reflecting mirror 1005, lenses 1006 and 1007, CCD imaging devices 1008 and 1009, an arithmetic operation unit 1010, and a monitor 1011.

The infrared light source 1001 irradiates infrared light from a certain side surface of a nail 1102 of a finger 1101. The visible light sources 1002 and 1003 irradiate visible light from a side surface on the opposite side to the certain side surface of the nail 1102 of the finger 1101. The dichroic mirror 1004 transmits the infrared light that passes through the finger 1101, and reflects the visible light that is reflected at the finger 1101. The reflecting mirror 1005 reflects the visible light that is reflected by the dichroic mirror 1004. The lens 1006 forms an image of the finger 1101 based on the visible light on the CCD imaging device 1008. The lens 1007 forms an image of the finger 1101 based on the infrared light on the CCD imaging device 1009. The CCD imaging device 1008 converts the formed visible light image into an image electric signal. The CCD imaging device 1009 converts the formed infrared light image into an image electric signal. The arithmetic operation unit 1010 performs signal processing for reducing the influence of dirt, wrinkles, or the like of a surface of the finger 1101 by using the visible light image and the infrared light image, and performs image processing in order to extract a vein pattern. The monitor 1011 displays the images of the finger 1101 which are captured by the CCD imaging devices 1008 and 1009 and the vein pattern of the finger 1101 which is extracted by the arithmetic operation unit 1010 as images.

According to the above-mentioned configuration, among light beams that are incident on the dichroic mirror 1004, infrared light passes through the dichroic mirror 1004 and is then incident on the lens 1006. On the other hand, visible light is reflected by the dichroic mirror 1004, is further reflected by the reflecting mirror 1005, and is separated from the infrared light, and is then incident on the lens 1007. Thus, it is possible to detect the vein pattern of the finger 1101 with a high level of accuracy without being influenced by dirt, wrinkles, or the like on the finger 1101.

Next, there is described a configuration of an apparatus used to detect a visible light image and an infrared light image in a vein authentication system, known in the related art, which is different from the example illustrated in FIG. 21. FIG. 22 is a schematic diagram illustrating a configuration of a vein authentication system, known in the related art, which detects a vein pattern by detecting a visible light image and an infrared light image. A vein authentication system 2000 includes an infrared light source 2001, visible light sources 2002 and 2003, a lens 2004, a CCD imaging device 2005, an arithmetic operation unit 2006, and a monitor 2007.

The vein authentication system 2000 illustrated in FIG. 22 has the same configuration as the configuration of the vein authentication system 1000 illustrated in FIG. 21 except for the dichroic mirror 1004 and the reflecting mirror 1005 that are respectively arranged on the input sides of the lenses 1006 and 1007. In addition, the vein authentication system 2000 alternately captures the infrared light image and the visible light image by alternately lighting the infrared light source 2001 and the visible light sources 2002 and 2003.

For example, first, the infrared light source 2001 is turned on to turn off the visible light sources 2002 and 2003, and the infrared light image passing through a finger 2101 is captured. Next, the infrared light source 2001 is turned off to turn on the visible light sources 2002 and 2003, and the visible light image that is reflected at the finger 2101 is captured. The arithmetic operation unit 2006 performs image processing for extracting a vein pattern by using the visible light image and the infrared light image. According to this configuration, it is possible to detect the vein pattern of the finger 2101 with a high level of accuracy without being influenced by dirt, wrinkles, or the like of the finger 2101.

In addition, Japanese Unexamined Patent Application, First Publication No. H10-201707 discloses an example of an endoscope system that detects cancer of biological tissue by irradiating the biological tissue with infrared light in addition to visible light and by using a visible light image and an infrared light image which are reflected by the biological tissue. Specifically, Japanese Unexamined Patent Application, First Publication No. H10-201707 discloses an example of acquiring an RGB visible light image and an infrared light image according to an ICG fluorescence component, and an example of sequentially acquiring an RGB visible light image and an infrared light image according to an ICG fluorescence component while rotating a transmission band filter and an RGB rotating filter, on the basis of a configuration using a dichroic mirror.

### SUMMARY OF THE INVENTION

According to a first aspect of the present invention, an imaging device includes a first filter that has a transmission band transmitting a light of a visible region and an infrared region; a first substrate that is arranged below the first filter, and has a first photoelectric conversion element which outputs a first signal charge according to an amount of exposure of a light passing through the first filter; a second substrate that has a second photoelectric conversion element outputting a second signal charge according to an amount of exposure of a light, having sensitivity in at least the infrared region, which passes through the first substrate, and is arranged on a surface on an opposite side to a light-receiving surface of the first substrate; and a signal read-out circuit that reads out the first signal charge as a first electrical signal, and reads out the second signal charge as a second electrical signal.

According to a second aspect of the present invention, in the above-mentioned first aspect, a size of a pixel including the second photoelectric conversion element may be an integer times a size of a pixel including the first photoelectric conversion element.

According to a third aspect of the present invention, in the above-mentioned first aspect, the first filter may include a filter that transmits a plurality of types of light beams of the infrared region.

According to a fourth aspect of the present invention, in the above-mentioned first aspect, the imaging device may further include a second filter that is arranged between the first substrate and the second substrate and shields the light of the visible region.

According to a fifth aspect of the present invention, in the above-mentioned first aspect, the imaging device may further include a correction circuit that reduces the influence on the first signal charge which derives from the light of the infrared region, by using the second electrical signal.

According to a sixth aspect of the present invention, in the above-mentioned first aspect, the imaging device may further include a supporting base that supports a finger; an illumination system that irradiates the supporting base with a light having a spectral distribution in the visible region and the infrared region; and an optical system that guides a light passing through the finger, which is supported by the supporting base, and a light reflected at the finger to the first substrate.

According to a seventh aspect of the present invention, in the above-mentioned first aspect, the imaging device may further include an illumination system that irradiates a subject with a light having spectral distribution in the visible region and an excitation light exiting fluorescence which has spectral distribution in the infrared region; and an optical system that guides a light from the subject to the first substrate.

### BRIEF DESCRIPTION OF THE DRAWINGS

FIG. 1 is a cross-sectional view illustrating a cross-section of an imaging device according to a first embodiment of the present invention.

FIG. 2 is a schematic diagram illustrating arrangement of pixels that are included in a first substrate having a color filter formed therein, according to the first embodiment of the present invention.

FIG. 3 is a schematic diagram illustrating arrangement of pixels that are included in a second substrate, according to the first embodiment of the present invention.

FIG. 4 is a schematic diagram illustrating an arrangement relationship between pixels of a set of unit pixel regions which are included in the first substrate and the pixels that are included in the second substrate, according to the first embodiment of the present invention.

FIG. 5 is a graph illustrating a transmission characteristic of a color filter according to the first embodiment of the present invention.

FIG. 6 is a cross-sectional view illustrating a cross-section of an imaging device with a supporting layer interposed between the first substrate and the second substrate, according to the first embodiment of the present invention.

FIG. 7 is a schematic diagram illustrating an arrangement relationship between pixels that are included in a first substrate and pixels that are included in a second substrate, according to a second embodiment of the present invention.

FIG. 8 is a schematic diagram illustrating an arrangement relationship between the pixels that are included in the first substrate and the pixels that are included in the second substrate, according to the second embodiment of the present invention.

FIG. 9 is a graph illustrating a transmission characteristic of a color filter according to a third embodiment of the present invention.

FIG. 10 is a schematic diagram illustrating an arrangement relationship between pixels of a set of unit pixel regions which are included in a first substrate and pixels that are included in a second substrate, according to the third embodiment of the present invention.

FIG. 11 is a cross-sectional view illustrating a cross-section of an imaging device according to a fourth embodiment of the present invention.

FIG. 12 is a schematic diagram illustrating first electrical signals that are output from pixels of a first substrate and second electrical signals that are output from pixels of a second substrate, according to a fifth embodiment of the present invention.

FIG. 13 is a schematic diagram illustrating a correction method using a correction circuit according to the fifth embodiment of the present invention.

FIG. 14 is a schematic diagram illustrating a configuration of a vein authentication system that detects a vein pattern by detecting a visible light image and an infrared light image, according to a sixth embodiment of the present invention.

FIG. 15 is a schematic diagram illustrating a configuration of an endoscope system that detects a specific portion by detecting a visible light image and an infrared light image, according to a seventh embodiment of the present invention.

FIG. 16 is a graph illustrating a transmission characteristic of a bandpass filter according to the seventh embodiment of the present invention.

FIG. 17 is a graph illustrating a transmission characteristic of an excitation light cut filter according to the seventh embodiment of the present invention.

FIG. 18 is a graph illustrating excitation and a fluorescence characteristic of indocyanine green according to the seventh embodiment of the present invention.

FIG. 19 is a graph illustrating spectral distribution detected by pixels of a first substrate that is included in an imaging device according to the seventh embodiment of the present invention.

FIG. 20 is a graph illustrating spectral distribution detected by pixels of a second substrate that is included in the imaging device according to the seventh embodiment of the present invention.

FIG. 21 is a schematic diagram illustrating a configuration of a vein authentication system, known in the related art, which detects a vein pattern by detecting a visible light image and an infrared light image.

FIG. 22 is a schematic diagram illustrating a configuration of a vein authentication system, known in the related art, which detects a vein pattern by detecting a visible light image and an infrared light image.

### DETAILED DESCRIPTION OF THE INVENTION

### First Embodiment

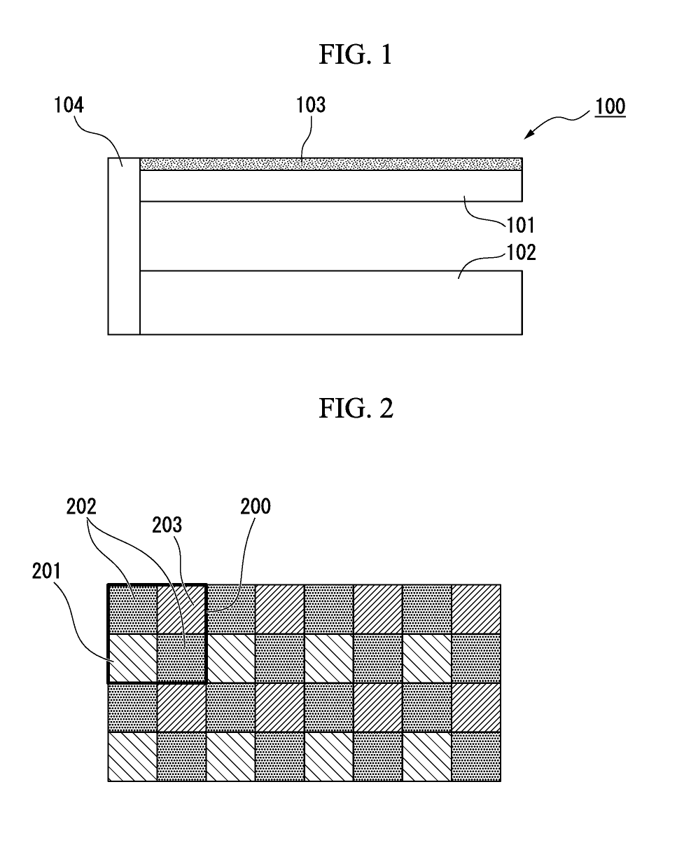

Hereinafter, a first embodiment of the present invention will be described with reference to the accompanying drawings. FIG. 1 is a cross-sectional view illustrating a cross-section of an imaging device 100 according to this embodiment. In the example shown in FIG. 1, the imaging device 100 includes a first substrate 101, a second substrate 102, a color filter 103 (first filter), and a connection unit 104. The first substrate 101 and the second substrate 102 are formed on a silicon chip and include a plurality of pixels. The RGB color filter 103 is formed on the light-receiving surface side of the first substrate 101. The arrangement of the color filter 103 and a wavelength of light that passes through the color filter 103 will be described later.

The color filter 103 is generated using an organic material (pigment). The color filter 103 has a feature that the red color filter 103 transmits red visible light and red infrared light, the green color filter 103 transmits green visible light and green infrared light, and the blue color filter 103 transmits blue visible light and blue infrared light.

In addition, the first substrate 101 and the second substrate 102 are laminated (stacked) on each other. In FIG. 1, the second substrate 102 is arranged on a surface opposite to the light-receiving surface of the first substrate 101. A light-receiving surface of the second substrate 102 is the side on which the first substrate 101 is present. In addition, the connection unit 104 is configured between the first substrate 101 and the second substrate 102, and the first substrate 101 and the second substrate 102 are electrically connected to each other through the connection unit 104. That is, the first substrate 101 and the second substrate 102 are bonded to each other through the connection unit 104.

Herein, the first substrate 101 is an imaging substrate of a rear surface irradiation type, and a thickness of the first substrate 101 is as small as approximately several um. For this reason, some of light beams that are incident from the light-receiving surface side of the first substrate 101 pass and are then incident on the light-receiving surface side of the second substrate 102. Meanwhile, the rate of absorption of light for each depth of silicon varies according to wavelengths. In a shallow portion of silicon, the rate of absorption of light having a short wavelength is high, and the rate of absorption of light having a long wavelength is low. That is, the first substrate 101 having a small thickness absorbs light having a short wavelength and does not absorb light having a long wavelength. For this reason, the first substrate 101 absorbs only visible light and transmits infrared light. Therefore, infrared light is incident on the second substrate 102. The second substrate 102 is a surface irradiation type imaging substrate, and the thickness of the second substrate 102 is larger than the thickness of the first substrate 101. For this reason, the infrared light that passes through the first substrate 101 is detected in the second substrate 102. The first substrate 101 is not limited to the rear surface irradiation type imaging substrate, and may be any substrate as long as it is a substrate that transmits infrared light.

FIG. 2 is a schematic diagram illustrating arrangement of pixels that are included in the first substrate 101 having the color filter 103 formed therein, according to this embodiment. FIG. 2 illustrates an example of a total of 32 pixels that are arranged in a two-dimensional shape of 4 rows 8 columns. The number and arrangement of the pixels that are included in the first substrate 101 are not limited to the example illustrated in the drawing, and any number and arrangement thereof may be employed.

In this embodiment, the arrangement of the color filter 103 is a Bayer array, and four pixels that are vertically and horizontally adjacent to each other are one set of unit pixel regions 200. For this reason, as illustrated in FIG. 2, the one set of unit pixel regions 200 include one pixel 201 in which the color filter 103 transmitting wavelength ranges of red light and infrared light is formed, two pixels 202 in which the color filter 103 transmitting wavelength ranges of green light and infrared light is formed, and one pixel 203 in which the color filter 103 transmitting wavelength ranges of blue light and infrared light is formed.

Each of the pixels 201 to 203 included in the first substrate 101 includes a photoelectric conversion element (first photoelectric conversion element) and a signal read-out circuit. Each photoelectric conversion element outputs a first signal charge according to an amount of exposure of light to the read-out circuit. The signal read-out circuit outputs the first signal charge, which is output from the photoelectric conversion element, as a first electrical signal.

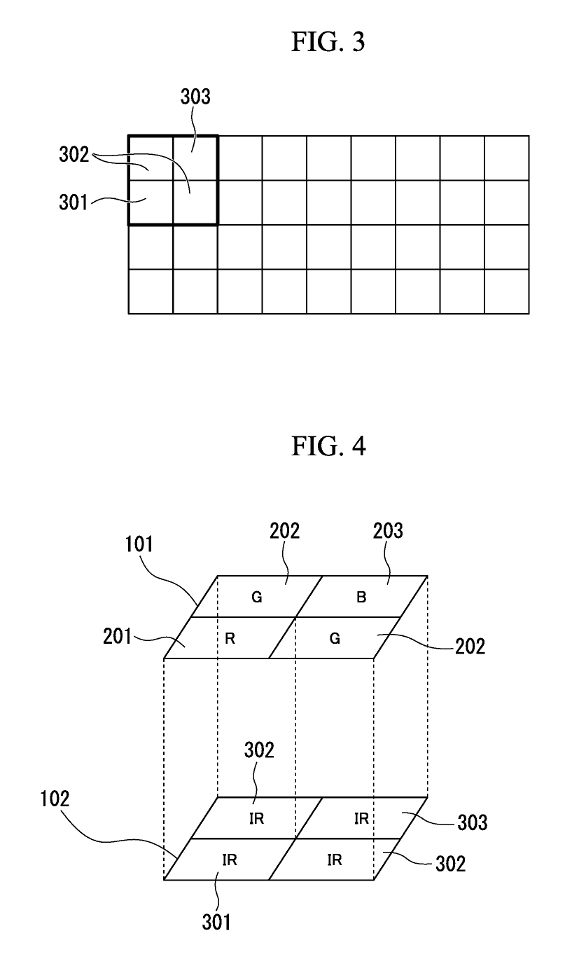

FIG. 3 is a schematic diagram illustrating the arrangement of pixels that are included in the second substrate 102 in this embodiment. FIG. 3 illustrates an example of a total of 32 pixels that are arranged in a two-dimensional shape of 4 rows 8 columns. Meanwhile, the number and arrangement of the pixels that are included in the second substrate 102 are not limited to the example illustrated in the drawing, and any number and arrangement thereof may be employed.

Each of pixels 301 to 303 included in the second substrate 102 includes a photoelectric conversion element (second photoelectric conversion element) and a signal read-out circuit. Each photoelectric conversion element outputs a second signal charge according to an amount of exposure of light to the read-out circuit. The signal read-out circuit outputs the second signal charge, which is output from the photoelectric conversion element, as a second electrical signal.

FIG. 4 is a schematic diagram illustrating an arrangement relationship between the pixels 201 to 203 of the one set of unit pixel regions 200 which are included in the first substrate 101 and the pixels 301 to 303 that are included in the second substrate 102 in this embodiment. In FIG. 4, the pixel 301 is arranged at a position on which infrared light passing through the pixel 201, provided with the color filter 103 transmitting red light and infrared light, is incident. In addition, the pixel 302 is arranged at a position on which infrared light passing through the pixel 202, provided with the color filter 103 transmitting green light and infrared light, is incident. In addition, the pixel 303 is arranged at a position on which infrared light passing through the pixel 203, provided with the color filter 103 transmitting blue light and infrared light, is incident. That is, the pixels 201 to 203 included in the first substrate 101 and the pixels 301 to 303 included in the second substrate 102 are associated with each other on a one-to-one basis.

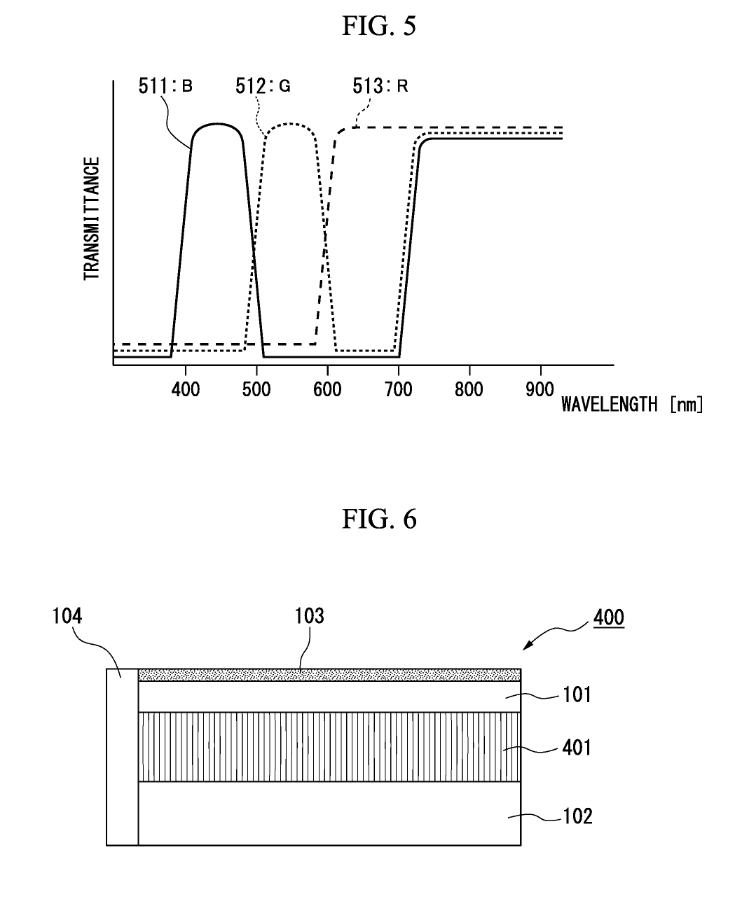

Next, a wavelength of light that passes through the color filter 103 will be described. FIG. 5 is a graph illustrating a transmission characteristic of the color filter 103 according to this embodiment. In the graph shown in the drawing, a horizontal axis represents a wavelength, and a vertical axis represents the transmittance of the color filter 103 in each wavelength. In FIG. 5, a line 511 indicates that the blue color filter 103 transmitting blue light and infrared light transmits light (blue light) having a wavelength of approximately 400 nm to 500 nm and light (infrared light) having a wavelength of equal to or greater than approximately 700 nm. In addition, a line 512 indicates that the green color filter 103 transmitting green light and infrared light transmits light (green light) having a wavelength of approximately 500 nm to 600 nm and light (infrared light) having a wavelength of equal to or greater than approximately 700 nm. In addition, a line 513 indicates that the red color filter 103 transmitting red light and infrared light transmits light (red light and infrared light) having a wavelength of equal to or greater than approximately 600 nm.

Next, an operation of the imaging device 100 will be described. In this embodiment, illumination light having a wavelength ranging from a visible region to an infrared region is used as a light source. An object such as biological tissue or a finger is irradiated with illumination light, and the transmitted light or reflected light thereof is incident on the imaging device 100.

Light is incident on the light-receiving surface side of the first substrate 101 in which the color filter 103 is formed. Like the transmission characteristic illustrated in FIG. 5, the red color filter 103 transmits red light and infrared light. In addition, the green color filter 103 transmits green light and infrared light. In addition, the blue color filter 103 transmits blue light and infrared light.

The pixels 201 to 203 of the first substrate 101 detect visible light beams that pass through the respective color filters 103, and output the first electrical signal. Specifically, the pixel 201 having the red color filter 103 formed therein outputs the first electrical signal in response to red light. In addition, the pixel 202 having the green color filter 103 formed therein outputs the first electrical signal in response to green light. In addition, the pixel 203 having the blue color filter 103 formed therein outputs the first electrical signal in response to blue light. A processing unit not shown in the drawing generates a visible light image on the basis of the first electrical signals that are output from the pixels 201 to 203.

The infrared light passing through the first substrate 101 is incident on the second substrate 102. Each of the pixels 301 to 303 of the second substrate 102 outputs the second electrical signal according to light having a wavelength of the infrared light. A processing unit not shown in the drawing generates an infrared light image on the basis of the second electrical signals that are output from the pixels 301 to 303.

As described above, according to this embodiment, the first substrate 101 and the second substrate 102 are laminated on each other. In addition, the first substrate 101 transmits infrared light. Thus, the pixels 201 to 203 included in the first substrate 101 can output the first electrical signal based on visible light. In addition, the pixels 301 to 303 included in the second substrate 102 can output the second electrical signal based on infrared light. In addition, it is possible to generate a visible light image on the basis of the first electrical signal and to generate an infrared light image on the basis of the second electrical signal.

In addition, in this embodiment, since light having a wavelength ranging from a visible region to an infrared region is used as a light source, temporal switching between visible light and infrared light is not necessary. For this reason, the imaging device 100 can simultaneously output the first electrical signal capable of generating the visible light image and the second electrical signal capable of generating the infrared light image. In addition, the imaging device 100 according to this embodiment does not require a dichroic mirror, a plurality of lenses, and imaging devices used to detect visible light and infrared light. For this reason, it is possible to achieve the miniaturization of the device and cost reduction. Therefore, according to this embodiment, the imaging device 100 can simultaneously acquire the visible light image and the infrared light image at a low price.

In order to increase the strength of the imaging device 100 that is constituted by the first substrate 101, the second substrate 102, the color filter 103, and the connection unit 104, a supporting layer may be interposed between the first substrate 101 and the second substrate 102. FIG. 6 is a cross-sectional view illustrating a cross-section of the imaging device in which a supporting layer is interposed between the first substrate 101 and the second substrate 102. In FIG. 6, an imaging device 400 includes the first substrate 101, the second substrate 102, the color filter 103, the connection unit 104, and a supporting layer 401. In addition, the supporting layer 401 is interposed between the first substrate 101 and the second substrate 102. The supporting layer 401 is required not to absorb light, to have conductivity, and to maintain a constant strength. A transparent conductive material such as indium tin oxide (ITO) is used as a material of the supporting layer 401. According to this configuration, it is possible to further increase the strength of the imaging device 400.

### Second Embodiment

Next, a second embodiment of the present invention will be described. This embodiment is different from the first embodiment in terms of a size of the pixel 301 included in the second substrate 102. Meanwhile, other configurations and operations are the same as those of the first embodiment.

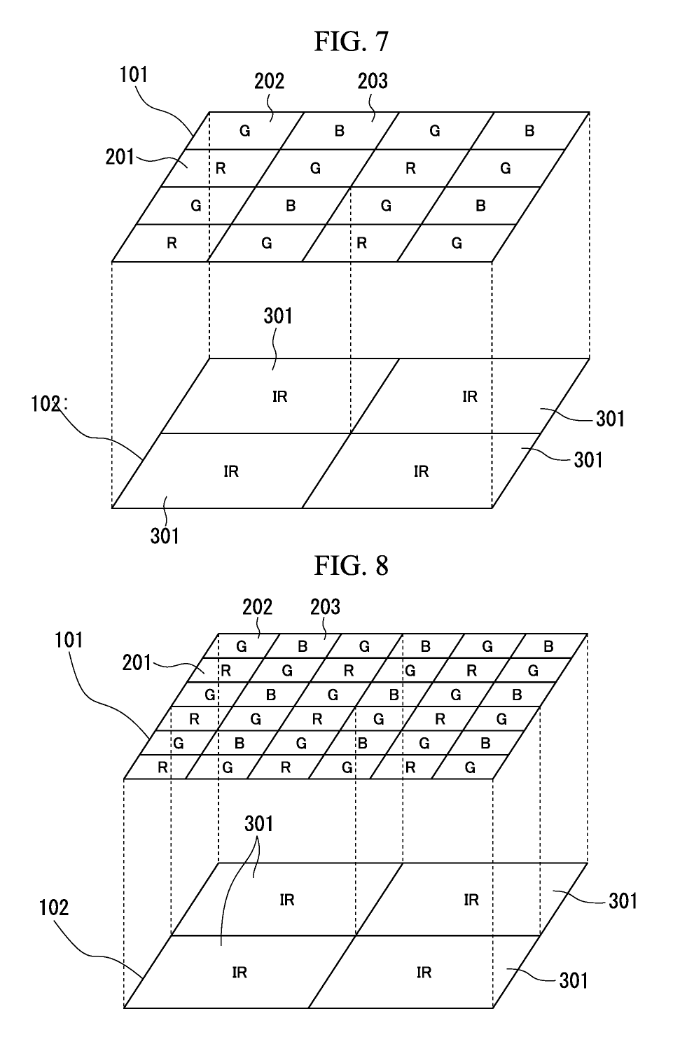

Hereinafter, an arrangement relationship between the pixels 201 to 203 that are included in the first substrate 101 and the pixels 301 that are included in the second substrate 102 according to this embodiment will be described. FIG. 7 is a schematic diagram illustrating the arrangement relationship between the pixels 201 to 203 that are included in the first substrate 101 and the pixels 301 that are included in the second substrate 102 in this embodiment. In the example shown in the drawing, one pixel 301 is arranged at a position on which infrared light passing through four pixels 201 to 203, which are included in one set of unit pixel regions 200, is incident. That is, the pixels 201 to 203 that are included in the first substrate 101 and the pixels 301 that are included in the second substrate 102 are associated with each other on a four-to-one basis.

The arrangement relationship between the pixels 201 to 203 that are included in the first substrate 101 and the pixels 301 that are included in the second substrate 102 is not limited to the example illustrated in FIG. 7, and any arrangement relationship may be employed as long as an integer number of pixels 201 to 203 included in the first substrate 101 correspond to one pixel 301 included in the second substrate 102. That is, any arrangement relationship may be employed as long as the size of each of the pixels 301 to 303 included in the second substrate 102 is an integer times the size of each of the pixels 201 to 203 included in the first substrate 101. For example, an arrangement relationship illustrated in FIG. 8 may be employed.

FIG. 8 is a schematic diagram illustrating an arrangement relationship between the pixels 201 to 203 that are included in the first substrate 101 and the pixels 301 that are included in the second substrate 102 in this embodiment. In the example shown in the drawing, one pixel 301 is arranged at a position on which infrared light passing through nine pixels 201 to 203 of three vertical columns and three horizontal columns, which are adjacent to each other, is incident. That is, the pixels 201 to 203 that are included in the first substrate 101 and the pixels 301 that are included in the second substrate 102 are associated with each other on a nine-to-one basis.

As described above, according to this embodiment, each of the pixels 301 that are included in the second substrate 102 detects a region that is larger than each of the pixels 201 to 203 that are included in the first substrate 101. For this reason, the amount of infrared light incident on each of the pixels 301 that are included in the second substrate 102 according to this embodiment increases as compared to each of the pixels 301 to 303 that are included in the second substrate 102 according to the first embodiment. Therefore, an SN ratio of infrared light that is detected in each of the pixels 301 that are included in the second substrate 102 increases, and thus it is possible to detect the infrared light with a high level of accuracy.

### Third Embodiment

Next, a third embodiment of the present invention will be described. This embodiment is different from the first embodiment in terms of a transmission characteristic of the color filter 103 that is formed in the first substrate 101. Meanwhile, other configurations and operations are the same as those of the first embodiment.

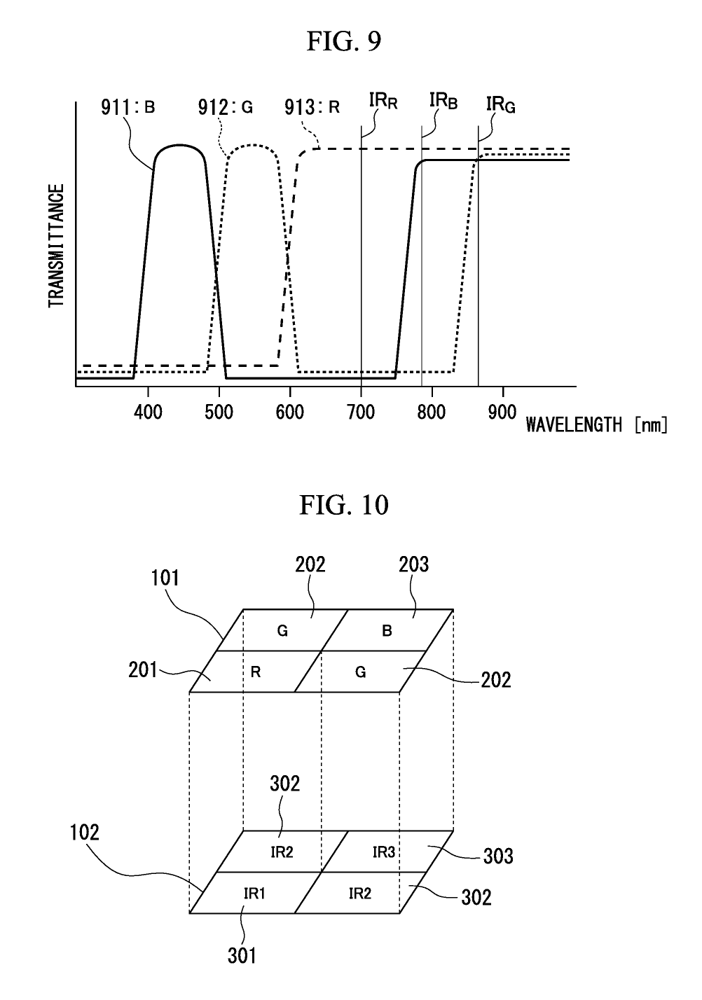

FIG. 9 is a graph illustrating a transmission characteristic of the color filter 103 according to this embodiment. In the graph shown in FIG. 9, a horizontal axis represents a wavelength, and a vertical axis represents the transmittance of the color filter 103 in each wavelength. In the example shown in FIG. 9, a line 911 indicates that the blue color filter 103 transmitting blue light and infrared light transmits light (blue light) having a wavelength of approximately 400 nm to 500 nm and light (infrared light) having a wavelength of equal to or greater than approximately 750 nm. In addition, a line 912 indicates that the green color filter 103 transmitting green light and infrared light transmits light (green light) having a wavelength of approximately 500 nm to 600 nm and light (infrared light) having a wavelength of equal to or greater than approximately 850 nm. In addition, a line 913 indicates that the green color filter 103 transmitting red light and infrared light transmits light (red light and infrared light) having a wavelength of equal to or greater than approximately 600 nm.

As illustrated in FIG. 9, in this embodiment, the blue color filter 103, the green color filter 103, and the red color filter 103 are different from each other in wavelength which initially rises in an infrared region. Specifically, in the infrared region, the transmittance of the blue color filter 103 rises at a wavelength (approximately 780 nm) of IR_{B}, the transmittance of the green color filter 103 rises at a wavelength (approximately 860 nm) of IR_{G}, and the transmittance of the red color filter 103 rises at a wavelength (approximately 700 nm) of IR_{R}.

FIG. 10 is a schematic diagram illustrating an arrangement relationship between the pixels 201 to 203 of one set of unit pixel regions 200 which are included in the first substrate 101 and the pixels 301 to 303 that are included in the second substrate 102 in this embodiment. In FIG. 10, the pixel 301 is arranged at a position on which infrared light passing through the pixel 201, provided with the color filter 103 transmitting red light and infrared light, is incident. In addition, the pixel 302 is arranged at a position on which infrared light passing through the pixel 202, provided with the color filter 103 transmitting green light and infrared light, is incident. In addition, the pixel 303 is arranged at a position on which infrared light passing through the pixel 203, provided with the color filter 103 transmitting blue light and infrared light, is incident. That is, the pixels 201 to 203 that are included in the first substrate 101 and the pixels 301 to 303 that are included in the second substrate 102 are associated with each other on a one-to-one basis.

In this embodiment, since the color filters 103 include the blue color filter 103, the green color filter 103, and the red color filter 103 that transmit a plurality of types of light beams of an infrared region, a wavelength of infrared light to pass varies depending on the color filters 103. For this reason, infrared light components that are detected by the pixels 301 to 303 included in the second substrate 102 are different from each other. Specifically, the pixel 301 detects an infrared light component IR1 having a wavelength that is longer than IR_{R}. In addition, the pixel 302 detects an infrared light component IR2 having a wavelength that is longer than IR_{G}. In addition, the pixel 303 detects an infrared light component IR3 having a wavelength that is longer than IR_{B}.

In this manner, according to this embodiment, the blue color filter 103, the green color filter 103, and the red color filter 103 are different from each other in transmission characteristic. Thus, the pixel 301 that is arranged at a position on which infrared light passing through the pixel 201 provided with the red color filter 103 is incident, the pixel 302 that is arranged at a position on which infrared light passing through the pixel 202 provided with the green color filter 103 is incident, and the pixel 303 that is arranged at a position on which infrared light passing through the pixel 203 provided with the blue color filter 103 is incident have different infrared light components incident thereon. Therefore, the pixels 301 to 303 that are included in the second substrate 102 can respectively detect infrared light components of different wavelength ranges.

In addition, it is possible to calculate only infrared light components in a predetermined range by using signals of the infrared light components IR1, IR2, and IR3 that are detected by the pixels 301 to 303. For example, it is possible to calculate only infrared light components in ranges of wavelengths IRB to IRG by calculating the infrared light components IR2 to IR3. In addition, for example, it is possible to calculate only the infrared light components in ranges of wavelengths IRR to IRB by calculating the infrared light components IR3 to IR1.

In this manner, it is possible to detect arbitrary infrared light components by mounting the color filters 103 having different characteristic of an infrared region and by arithmetically processing the second electrical signals that are detected by the pixels 301 to 303 of the second substrate 102.

### Fourth Embodiment

Next, a fourth embodiment of the present invention will be described. This embodiment is different from the first embodiment in that a visible light cut filter (second filter) is formed on the light-receiving surface side (between the first substrate 101 and the second substrate 102) of the second substrate 102. Meanwhile, other configurations and operations are the same as those of the first embodiment.

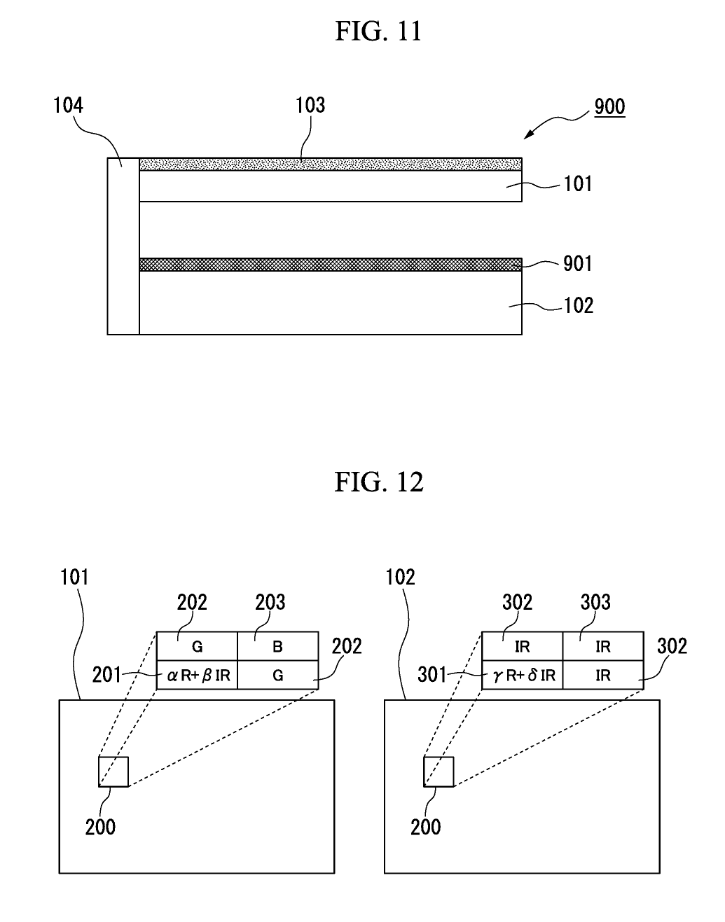

FIG. 11 is a cross-sectional view illustrating a cross-section of an imaging device 900 according to this embodiment. In FIG. 11, the imaging device 900 includes a first substrate 101, a second substrate 102, a color filter 103, a connection unit 104, and a visible light cut filter 901. The first substrate 101, the second substrate 102, the color filter 103, and the connection unit 104 are the same as those according to the first embodiment. The visible light cut filter 901 is a filter that absorbs visible light and transmits only infrared light. In addition, the visible light cut filter 901 is formed on the light-receiving surface side of the second substrate 102, that is, between the first substrate 101 and the second substrate 102.

In the first substrate 101, both blue light and green light which have a short wavelength are absorbed. For this reason, pixels 202 and 203 respectively provided with the blue color filter 103 and green color filter 103 transmit only infrared light. However, the first substrate 101 does not absorb all red light beams having a long wavelength, and transmits several percent of them. For this reason, each pixel 201 having the red color filter 103 formed therein transmits several percent of red light beams other than infrared light. Consequently, in this embodiment, the visible light cut filter 901 is provided between the first substrate 101 and the second substrate 102 so that light of a visible region is shielded and only light of an infrared region is incident on the second substrate 102.

Thus, since only infrared light is incident on the pixels 301 to 303 of the second substrate 102, the pixels 301 to 303 output a second electrical signal in response to only infrared light. Therefore, according to this embodiment, the imaging device 900 can output the second electrical signal in response to only infrared light without being influenced by red light.

### Fifth Embodiment

Next, a fifth embodiment of the present invention will be described. As described in the fourth embodiment, a first substrate 101 absorbs both blue light and green light which have a short wavelength. For this reason, pixels 203 and 202 respectively provided with a blue color filter 103 and a green color filter 103 transmit only infrared light. However, the first substrate 101 does not absorb all red light beams having a long wavelength, and transmits several percent of them. For this reason, each pixel 201 having a red color filter 103 formed therein transmits several percent of red light beams other than infrared light. Therefore, infrared light and several percent of red light beams are incident on a pixel 301 that is arranged at a position on which infrared light passing through the pixel 201 is incident. Accordingly, the pixel 301 outputs a second electrical signal in response to the infrared light and the several percent of red light beams.

In addition, there is a possibility of the pixel 201 provided with the red color filter 103 of the first substrate 101 absorbing some of infrared light beams. For this reason, the pixel 201 outputs a first electrical signal in response to read light and several percent of light beams having a wavelength of an infrared region.

Consequently, an imaging device according to this embodiment includes a correction circuit that corrects outputs of the pixel 201 and the pixel 301, and a memory unit that stores the first electrical signal and the second electrical signal which are output from the pixels 201 to 203 and the pixels 301 to 303, in order to exclude the influence of infrared light from the first electrical signal and to exclude the influence of red light from the second electrical signal. Meanwhile, the correction circuit and the memory unit may be included outside the imaging device rather than the inside thereof.

FIG. 12 is a schematic diagram illustrating the first electrical signals that are output from the pixels 201 to 203 of the first substrate 101 and the second electrical signals that are output from the pixels 301 to 303 of the second substrate 102 according to this embodiment. In FIG. 12, R denotes the intensity of red light. In addition, G denotes the intensity of green light. In addition, B denotes the intensity of blue light. In addition, IR denotes the intensity of infrared light. In addition, α denotes the ratio of red light which the pixel 201 absorbs. In addition, β denotes the ratio of infrared light which the pixel 201 absorbs. In addition, γ denotes the ratio of red light which the pixel 301 absorbs. In addition, δ denotes a ratio of infrared light which the pixel 301 absorbs.

Values of α, β, γ, and δ and relationships between α and γ and between β and δ can be calculated from the spectral sensitivity (sensitivity with respect to wavelength) of the first substrate 101 and the second substrate 102, and are parameters that are determined by a method (thicknesses, quantum efficiency, or the like of the first substrate 101 and the second substrate 102) of manufacturing an imaging device. The correction circuit stores the values of α, β, γ, and δ and the relationships between α and γ and between β and δ as information used for correction. Meanwhile, α, γ, β, and δ are real numbers equal to or greater than 0 and equal to or less than 1.

In FIG. 12, it is indicated that the pixel 201 having the red color filter 103 formed therein outputs αR+βIR. In addition, it is indicated that the pixel 202 having the green color filter 103 formed therein outputs G. In addition, it is indicated that the pixel 203 having the blue color filter 103 formed therein outputs B. In addition, it is indicated that the pixel 301 on which light passing through the pixel 201 is incident outputs γR+δIR. In addition, it is indicated that the pixel 302 on which light passing through the pixel 202 is incident outputs IR. In addition, it is indicated that the pixel 303 on which light passing through the pixel 203 is incident outputs IR.

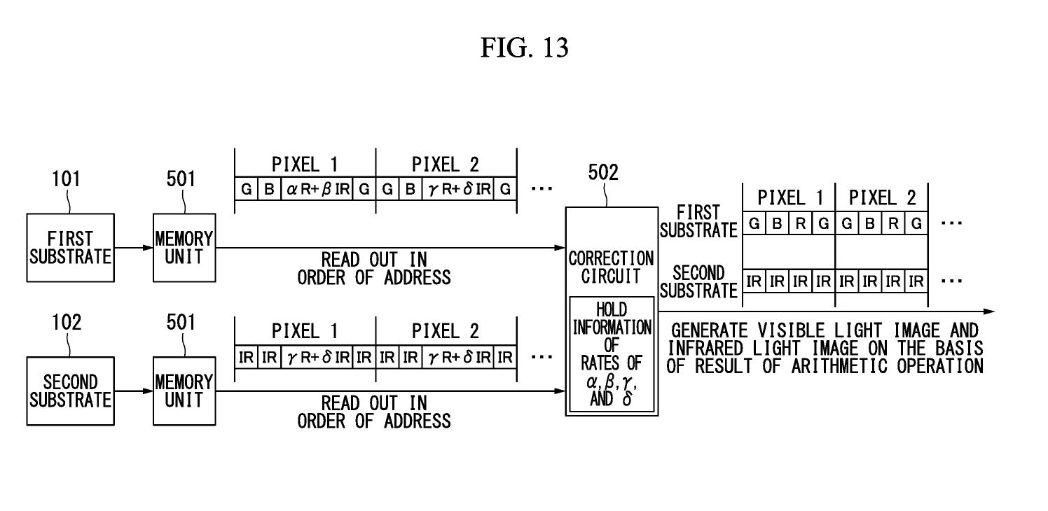

Next, a correction method using the correction circuit will be described. FIG. 13 is a schematic diagram illustrating the correction method using the correction circuit according to this embodiment. The pixels 201 to 203 of the first substrate 101 and the pixels 301 to 303 of the second substrate 102 output signals in response to the intensity of incident light. A memory unit 501 stores the signals that are output from the pixels 201 to 203 and the pixels 301 to 303. A correction circuit 502 sequentially reads out the signals that are output from the pixels 201 to 203 and the pixels 301 to 303 in order of address from the memory unit 501 and performs a correction process.

Hereinafter, an example of the correction process will be described. The correction circuit 502 calculates δIR, which is an output in a case where only infrared light is incident on the pixel 301 included in the second substrate 102, by performing an interpolating process using the pixels 302 and 303 that are adjacent to each other. For example, an average value of eight pixels 302 and 303 that are adjacent to the pixel 301 is set to δIR. Subsequently, the correction circuit 502 calculates βIR from the values of β and δ and the relationship between β and δ which are previously stored therein and the calculated δIR. The correction circuit 502 differentiates the calculated βIR from the first electrical signal (αR+βIR) that is output from the pixel 201 included in the first substrate 101. Thus, it is possible to calculate a pure red signal αR. Meanwhile, the correction process is not limited thereto, and any process may be employed as long as it is a process capable of calculating a pure red signal.

As described above, the correction circuit 502 corrects the second electrical signals that are output from the pixels 301 to 303 of the second substrate 102. Thus, it is possible to calculate the second electrical signal in response to light having only a wavelength of an infrared region without forming a visible light cut filter between the first substrate 101 and the second substrate 102. In addition, the correction circuit 502 corrects the first electrical signal that is output from the pixel 201 having the red color filter 103 formed therein, by using the corrected second electrical signal. Thus, it is possible to calculate only a pure red signal by excluding the influence of infrared light.

### Sixth Embodiment

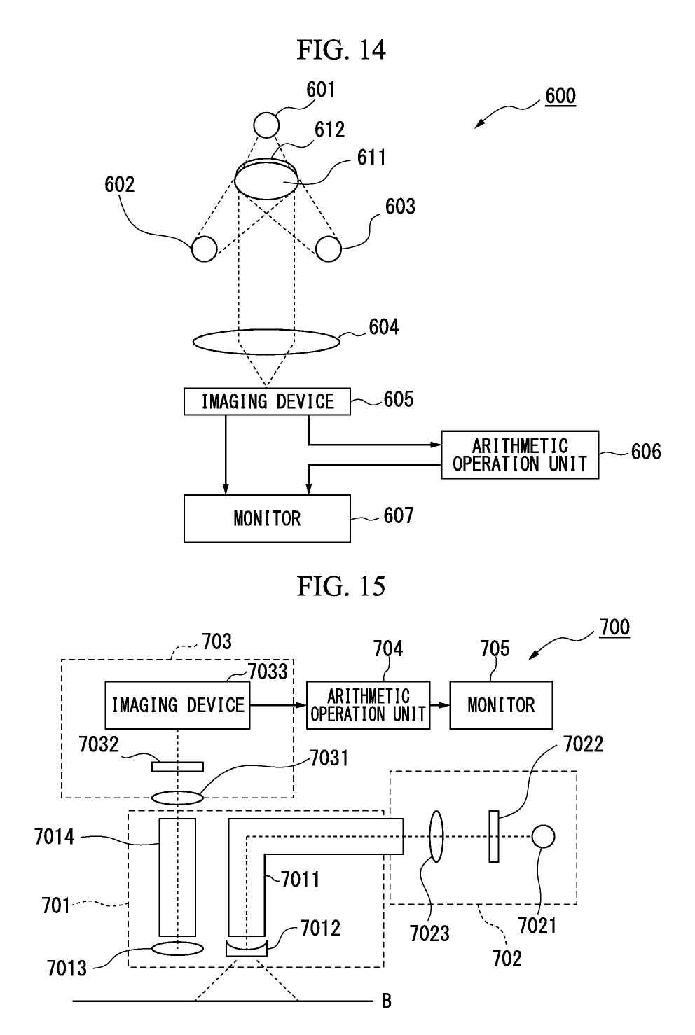

Next, a sixth embodiment of the present invention will be described. In this embodiment, an example will be described in which any one of the imaging devices described in the first embodiment to the fifth embodiment is mounted to a vein authentication system. FIG. 14 is a schematic diagram illustrating a configuration of the vein authentication system that detects a vein pattern by detecting a visible light image and an infrared light image according to this embodiment. A vein authentication system 600 includes an infrared light source 601, visible light sources 602 and 603, a lens 604, an imaging device 605, an arithmetic operation unit 606, and a monitor 607. In addition, the vein authentication system 600 includes a supporting base (not shown) which supports a finger.

The infrared light source 601 irradiates a supporting base, not shown in the drawing, with infrared light. Specifically, the infrared light source 601 irradiates the infrared light from a certain side surface of a nail 612 of a finger 611 which is supported by the supporting base not shown in the drawing. The visible light sources 602 and 603 irradiate the supporting base, not shown in the drawing, with visible light. Specifically, the visible light sources 602 and 603 irradiate the visible light from a side surface on the opposite side to the certain side surface of the nail 612 of the finger 611 which is supported by the supporting base not shown in the drawing. An image of the finger 611 based on the infrared light passing through the finger 611 and the visible light reflected at the finger 611 is formed on the imaging device 605. The imaging device 605 is any one of the imaging devices described in the first embodiment to the fifth embodiment. Pixels 201 to 203 included in a first substrate 101 of the imaging device 605 output a first electrical signal which is a visible light image of the finger 611 based on the visible light. In addition, pixels 301 to 303 included in a second substrate 102 of the imaging device 605 output a second electrical signal which is a vein pattern image of the finger 611 based on the infrared light.

The arithmetic operation unit 606 performs signal processing used to reduce the influence of dirt, wrinkles, or the like of a surface of the finger 611 by using the first electrical signal and the second electrical signal, and performs image processing in order to extract a vein pattern. The monitor 607 displays the image of the finger 611 which is captured by the imaging device 605 and the vein pattern of the finger 611 which is extracted by the arithmetic operation unit 606 as images.

According to the above-mentioned configuration, the vein authentication system 600 can simultaneously capture the visible light image and the infrared light image without including a dichroic mirror used to separate visible light and infrared light from each other and lenses used to respectively image the visible light and the infrared light that are separated from each other by the dichroic mirror. Therefore, it is possible to simultaneously capture the visible light image and the infrared light image while achieving miniaturization of the device and cost reduction.

### Seventh Embodiment

Next, a seventh embodiment of the present invention will be described. In this embodiment, an example will be described in which any one of the imaging devices described in the first embodiment to the fifth embodiment is mounted to an endoscope system. The endoscope system can determine the presence or absence of cancer by using an infrared light image. For example, there is a diagnosis method that administers in advance a fluorescent material having a predilection for cancer into the body of an object to be inspected and that irradiates the object with excitation light used to excite the fluorescent material to thereby detect fluorescence (infrared light) from the fluorescent materials accumulating in the cancer. Consequently, in this embodiment, the pixels 201 to 203 included in the first substrate 101 acquire a visible light image, and the pixels 301 to 303 included in the second substrate 102 acquire a fluorescence image (infrared light image) from the fluorescent material by using any one of the imaging devices described in the first embodiment to the fifth embodiment.

FIG. 15 is a schematic diagram illustrating a configuration of the endoscope system that detects a specific portion by detecting a visible light image and an infrared light image according this embodiment. An endoscope system 700 includes an endoscope unit 701 used to observe and diagnose the inside of a body, a light source unit 702 that emits light used in observation and light used in excitation, an imaging unit 703 that captures a visible light image and an infrared light image that are reflected by or emitted from a human body, an arithmetic operation unit 704 that performs signal processing of the captured visible light image and infrared light image, and a monitor 705 that displays an image.

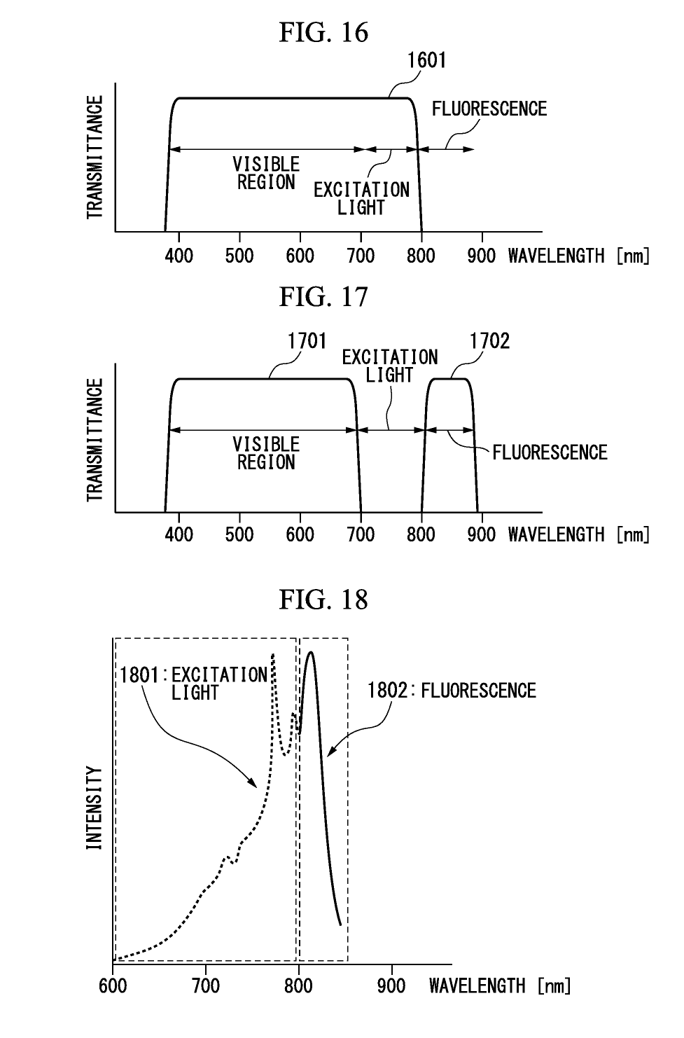

The light source unit 702 includes a light source 7021 that emits light including a wavelength ranging from a visible region, which includes a wavelength range of excitation light, to an infrared region, a bandpass filter 7022 that is provided in a light path of the light source 7021 and limits a transmission wavelength range, and a condenser lens 7023 used to condense light passing through the bandpass filter 7022. FIG. 16 is a graph illustrating a transmission characteristic of the bandpass filter 7022 according to this embodiment. In the graph shown in the drawing, a horizontal axis represents a wavelength, and a vertical axis represents the transmittance of the bandpass filter 7022 in each wavelength. In the example shown in the drawing, a line 1601 indicates that the bandpass filter 7022 transmits a light having a wavelength of approximately 400 nm to 800 nm which is a wavelength range including a visible region used in observation and an infrared region of excitation light.

Light from the light source 7021 is incident on a light guide 7011 of the endoscope unit 701 through the bandpass filter 7022 and the condenser lens 7023. A human body is irradiated with the light that is transmitted by the light guide 7011 from an illumination lens 7012 that is provided in a tip portion of the endoscope unit 701. The human body is irradiated with both visible light used in observation and excitation light used to observe observing fluorescence.

An object lens 7013 is provided in the tip portion of the endoscope unit 701 so as to be adjacent to the illumination lens 7012, and reflected light (visible region and infrared region of excitation light) from the human body and fluorescence (infrared region having a longer wavelength than excitation light) are incident on the object lens 7013. A tip surface of an image guide 7014 as a transmission unit of an optical image is arranged at an imaging position of the object lens 7013, and the optical image that is formed on the tip surface is transmitted to the imaging unit 703 side.

The optical image transmitted by the image guide 7014 is formed on an imaging device 7033 by an imaging lens 7031. An excitation light cut filter 7032 for removing an excitation light component from infrared light is arranged between the imaging lens 7031 and the imaging device 7033. FIG. 17 is a graph illustrating a transmission characteristic of the excitation light cut filter 7032 according to this embodiment. In the graph shown in the drawing, a horizontal axis represents a wavelength, and a vertical axis represents the transmittance of the excitation light cut filter 7032 in each wavelength. In the example shown in the drawing, lines 1701 and 1702 indicate that the excitation light cut filter 7032 transmits light having a wavelength of approximately 400 nm to 700 nm which is a visible region, and light having a wavelength of approximately 800 nm to 900 nm which is a wavelength range that is longer than a wavelength range of excitation light in an infrared region. Therefore, the wavelength range of the excitation light is removed by the excitation light cut filter 7032, and thus only visible light and fluorescence are incident on the imaging device 7033.

The pixels 201 to 203 of the first substrate 101 of the imaging device 7033 detect visible light beams (used in observation) passing through the respective color filters 103 and output a first electrical signal. Specifically, the pixel 201 having the red color filter 103 formed therein outputs the first electrical signal in response to light having a red wavelength. In addition, the pixel 202 having the green color filter 103 formed therein outputs the first electrical signal in response to light having a green wavelength. In addition, the pixel 203 having the blue color filter 103 formed therein outputs the first electrical signal in response to light having a blue wavelength. The imaging device 7033 generates a visible light image on the basis of the first electrical signals that are output from the pixels 201 to 203.

Infrared light (only a fluorescence component) passing through the first substrate 101 is incident on the second substrate 102 of the imaging device 7033. The pixels 301 to 303 of the second substrate 102 output a second electrical signal in response to light having a wavelength of infrared light. The imaging device 7033 generates a fluorescence image on the basis of the second electrical signals that are output from the pixels 301 to 303. The visible light image and the fluorescence image that are generated by the imaging device 7033 are input to the monitor 705. The monitor 705 displays the input visible light image and fluorescence image on a display surface.

An illumination system according to this embodiment is, for example, the light source unit 702, the light guide 7011, and the illumination lens 7012. In addition, an optical system according to this embodiment is, for example, the object lens 7013, the image guide 7014, and the imaging lens 7031.

Next, a procedure of performing diagnosis using the endoscope system 700 will be described. Indocyanine green is administered in advance into the body of an object to be inspected before performing diagnosis using the endoscope system 700. Since the indocyanine green has a predilection for cancer, the indocyanine green accumulates in a focus portion such as cancer when it is administered into the body and left to stand for a period of time.

FIG. 18 is a graph illustrating excitation and a fluorescence characteristic of indocyanine green according to this embodiment. In the graph shown in the drawing, a horizontal axis represents a wavelength, and a vertical axis represents the intensity of each wavelength. In FIG. 18, a line 1801 indicates the intensity of excitation light. In addition, a line 1802 indicates the intensity of fluorescence. As illustrated in FIG. 18, a peak wavelength of the excitation light is approximately 770 nm, and a peak wavelength of the fluorescence is approximately 810 nm. Therefore, the inside of the body is irradiated with light having a wavelength of approximately 770 nm to 780 nm, and then light having a wavelength of approximately 810 nm to 820 nm is detected, thereby detecting the presence or absence of cancer.

For this reason, the bandpass filter 7022 having a transmission characteristic which is illustrated in FIG. 15 is used so that a wavelength range of light with which a human body is irradiated includes light having a wavelength of approximately 770 nm to 780 nm and does not include light having a wavelength of approximately 810 nm to 820 nm. In addition, since the second substrate 102 of the imaging device 7033 detects only infrared light of a fluorescence component, light having a wavelength of 700 nm to 800 nm is cut (not transmitted).

Light from the light source 7021 passes through the bandpass filter 7022 to thereby become a light component including wavelength ranges of visible light and excitation light. The light passing through the bandpass filter 7022 is condensed by the condenser lens 7023 and is then incident on the light guide 7011. A human body B is irradiated with the light, which is transmitted by the light guide 7011, through the illumination lens 7012. In the human body B, illumination light is reflected, and fluorescence is emitted by indocyanine green being irradiated with excitation light. The reflected light and the fluorescence from the human body B are incident on the imaging device 7033 through the object lens 7013, the image guide 7014, the imaging lens 7031, and the excitation light cut filter 7032.

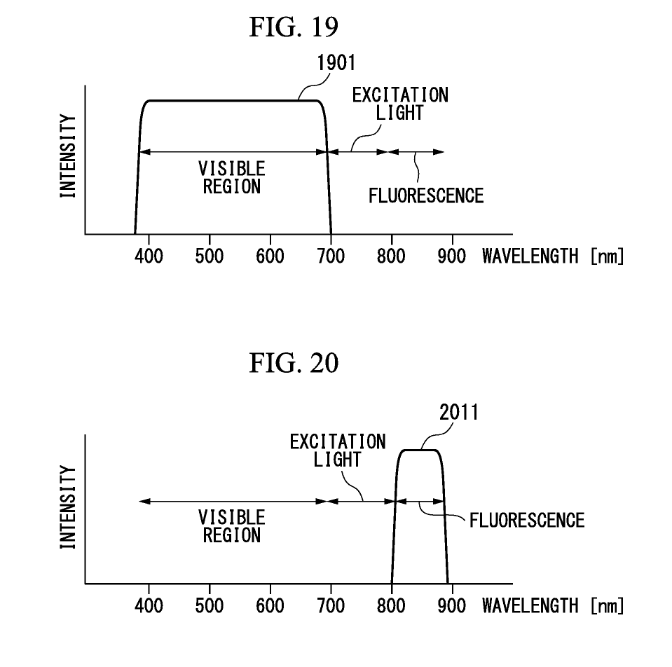

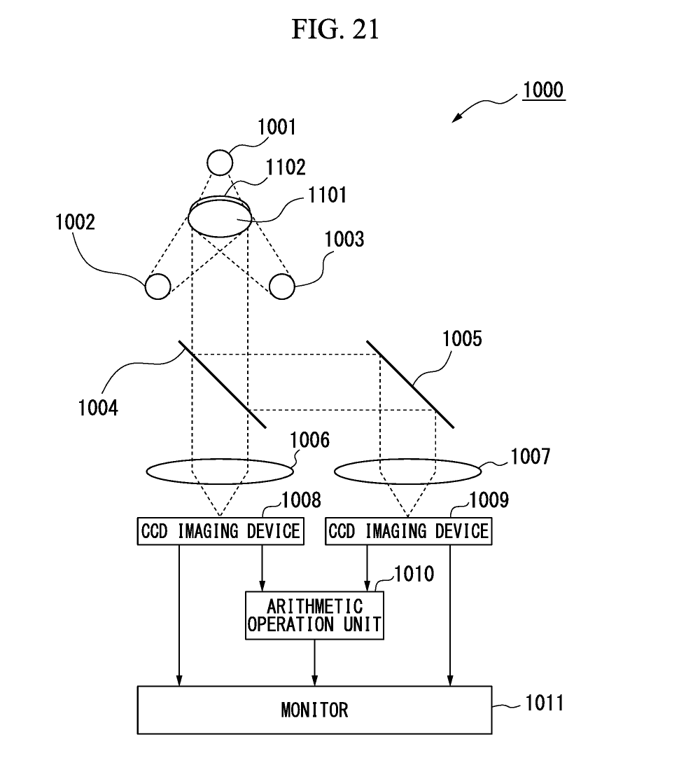

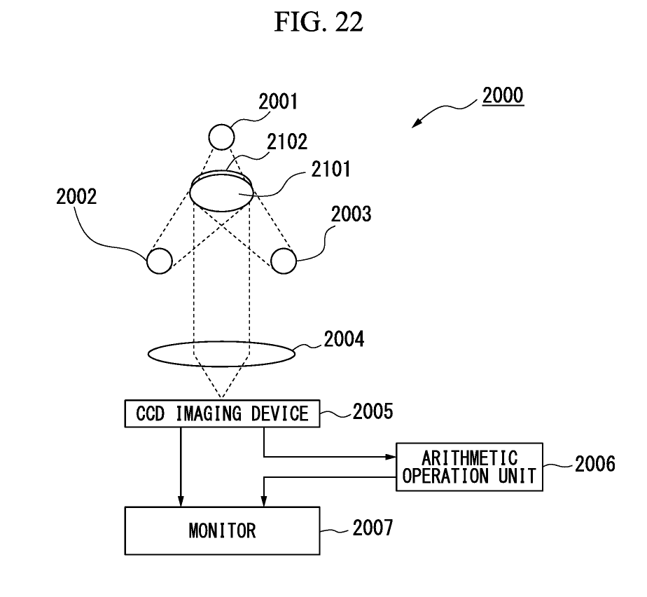

FIG. 19 is a graph illustrating spectral distribution detected by the pixels 201 to 203 of the first substrate 101 that is included in the imaging device 7033 in this embodiment. In the graph shown in the drawing, a horizontal axis represents a wavelength, and a vertical axis represents the sensitivity with respect to each of wavelengths that are detected by the pixels 201 to 203. A line 1901 indicates the sensitivity with respect to each of wavelengths that are detected by the pixels 201 to 203. The example shown in the drawing indicates that the pixels 201 to 203 detect light having a wavelength of approximately 400 nm to 700 nm which is a visible region. As described above, the imaging device 7033 generates a visible light image on the basis of the first electrical signals that are output from the pixels 201 to 203.

FIG. 20 is a graph illustrating spectral distribution detected by the pixels 301 to 303 of the second substrate 102 that is included in the imaging device 7033 in this embodiment. In the graph shown in the drawing, a horizontal axis represents a wavelength, and a vertical axis represents the intensity of light having each of wavelengths that are detected by the pixels 301 to 303. A line 2011 indicates the intensity of light having each wavelength which is detected by the pixels 301 to 303. The example shown in the drawing indicates that the pixels 301 to 303 detect light having a wavelength of approximately 800 nm to 900 nm which is a fluorescence range. As described above, the imaging device 7033 generates a fluorescence image on the basis of the second electrical signals that are output from the pixels 301 to 303.

The imaging device 7033 outputs the generated visible light image and fluorescence image to the monitor 705. The monitor 705 performs selection to display the visible light image and the fluorescence image which are input from the imaging device 7033 so as to be next to each other, or to display an image on which signal processing is performed using the visible light image and the infrared light image.

According to the above-mentioned configuration, the vein authentication system 700 can simultaneously capture a visible light image and an infrared light image without including a dichroic mirror used to separate visible light and infrared light (fluorescence) from each other and lenses used to respectively image the visible light and the infrared light which are separated from each other by the dichroic mirror. Therefore, it is possible to simultaneously capture the visible light image and the infrared light image while achieving the miniaturization of the device and cost reduction. In addition, since the visible light image and the infrared light image can be acquired simultaneously, it is possible to obtain the position of cancer in the visible light image simply and with a high level of accuracy, which results in usefulness at the time of performing diagnosis and medical treatment.

In addition, in general, wavelength ranges that are detected by the pixels 301 to 303 of the second substrate 102 are wide, and thus it is not possible to detect only an infrared light component of a specific wavelength range. However, when the imaging device 7033 of the endoscope system 700 is the imaging device 100 that is described in the third embodiment, it is possible to detect an arbitrary infrared light component by arithmetically processing the second electrical signals that are detected by the pixels 301 to 303 of the second substrate 102. In this manner, when light (light having a wavelength of approximately 810 nm to 820 nm) which has a relatively narrow wavelength range, like fluorescence, is detected by detecting only the infrared light component of the specific wavelength range, it is possible to remove an unnecessary infrared light component (light having a wavelength other than 810 nm to 820 nm with which a sensor is irradiated).

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An imaging device comprising:
a first filter that has a transmission band transmitting a light of a visible region and an infrared region;
a first substrate that is arranged below the first filter, and has a first photoelectric conversion element which outputs a first signal charge according to an amount of exposure of a light passing through the first filter;
a second substrate that has a second photoelectric conversion element outputting a second signal charge according to an amount of exposure of a light, having sensitivity in at least the infrared region, which passes through the first substrate, and is arranged on a surface on an opposite side to a light-receiving surface of the first substrate; and
a signal read-out circuit that reads out the first signal charge as a first electrical signal, and reads out the second signal charge as a second electrical signal.

2. The imaging device according to claim 1, wherein a size of a pixel including the second photoelectric conversion element is integer times a size of a pixel including the first photoelectric conversion element.

3. The imaging device according to claim 1, wherein the first filter includes a filter that transmits a plurality of types of light beams of the infrared region.

4. The imaging device according to claim 1, further comprising a second filter that is arranged between the first substrate and the second substrate and shields the light of the visible region.

5. The imaging device according to claim 1, further comprising a correction circuit that reduces influence on the first signal charge which derives from the light of the infrared region, by using the second electrical signal.

6. The imaging device according to claim 1, further comprising:
a supporting base that supports a finger;
an illumination system that irradiates the supporting base with a light having a spectral distribution in the visible region and the infrared region; and
an optical system that guides a light passing through the finger, which is supported by the supporting base, and a light reflected at the finger to the first substrate.

7. The imaging device according to claim 1, further comprising:
an illumination system that irradiates a subject with a light having spectral distribution in the visible region and an excitation light exiting fluorescence which has spectral distribution in the infrared region; and
an optical system that guides a light from the subject to the first substrate.
